(19) United States
(12) Patent Application Publication
(10) Pub. No.: US 2010/0145294 A1
(43) Pub. Date: Jun. 10, 2010

(54) Three-dimensional vertical hydration/dehydration sensor

(76) Inventors: Xuedong Song, Alpharetta, GA (US); James M. Takeuchi, Roswell, GA (US)

Correspondence Address: KIMBERLY-CLARK WORLDWIDE, INC.;Tara Pohlkotte, 2300 Winchester Rd., NEENAH, WI, 54956, US

(21) Appl. No.: 12/315,693

(22) Filed: Dec. 5, 2008

(51) Int. Cl. G01N 33/50; A61F 13/42; G01N 33/53

(52) U.S. Cl. 604/361; 422/56; 435/287.7; 422/69

(57) ABSTRACT

A three-dimensional fluidic assay device or sensor is described. The sensor has a porous substrate with a first face or plane defined along x-y coordinate axes, and a second face remote in a z-direction from the first face. The first face contains a sample deposition zone and the second face has at least one detection zone, such that when a fluid sample is deposited in the sample deposition zone, fluid is transported by means of capillary action along the z-direction to the detection zone and manifests a signal development. An absorbent article, such as a diaper or feminine hygiene product, having such a three-dimensional sensor integrated across the thickness of the absorbent article, from an inner layer to an outer layer, is also described.

### FIELD OF INVENTION

The present invention relates to a sensor and absorbent products containing the sensor. In particular, the invention pertains to a sensor that can monitor a user's hydration status.

### BACKGROUND

Dehydration is the depletion of fluids and associated electrolytes from the body. Normally, a person's daily, total fluid amount is regulated to be within about ±0.02% of body weight, and water in the body may comprise approximately 63% of the entire body mass. A balance of bodily fluids is achieved and maintained by matching the input and excretion of liquid from the body, and an imbalance in fluids can be linked to either dehydration or hypohydration. Dehydration can be of particular concern for either the infirm, elderly, or infants, and can have serious consequences to a dehydrated person if not cared for properly. Loss of body fluids in amounts of less than about 2-5% body mass have been associated with reduced heat dissipation, loss of cardiovascular function, and decreased physical stamina.

Specific gravity of an individual's urine is a routinely measured means of evaluating the relative hydration status of the individual. Specific gravity can be measured by an instrument such as either an urinometer or urine test dipsticks or strips, which are more commonly used nowadays. Determination of urine volume and electrolyte concentrations can aid in monitoring whether the individual's body fluid amounts are in balance.

Over the years, various manufacturers have attempted different methods to improve the performance of the dipsticks for specific gravity, such as different formulations to increase sensitivity and specificity. Two problems, however, have persisted for all the commercially available dipsticks. The first problem is that the user has to read a change in color within a few brief minutes after dipping in the sample because the color development is not stable under test conditions. The second problem is that since most of the reagents, such as color indicators are water soluble, the reagents can leach and test result can bleed and may be messy to read. Because of this second problem, test dipsticks require the user to dip and withdraw the dipstick quickly, or otherwise, reagents may leach into the sample. These two major problems of the existing technology have prevented the successful integration and commercialization of relatively low-cost hydration dipstick-type sensors in absorbent articles.

Currently, no device is available commercially that allows a caregiver or user to observe test results from outside of an absorbent article having any bulk or thickness, such as a diaper, adult incontinence or feminine hygiene products. This situation is due in part to technical difficulties of integrating hydration sensors into the bulk of an absorbent article where constant monitoring is not feasible.

Caregivers and parents are interested in an absorbent product (e.g., a diaper) that can monitor a wear's (e.g., baby's) hydration status. Such a product can assure caretakers and parents if the wear has taken enough fluid. Thus, a need exists for a sensor that can communicate pertinent test results through the bulk of the absorbent article in a cost effective way, and further, a need exists for an absorbent article incorporating such a sensor.

### SUMMARY OF THE INVENTION

The present invention pertains to a three-dimensional fluidic assay device or sensor having a porous substrate with a first face or plane, as defined along x-y coordinate axes, and a second face remote in a z-direction from the first face. The first face has a sample deposition zone. The second face has at least one detection zone, and in some embodiments as at least one control zone. When a sample is deposited in the sample deposition zone, fluid is transported by means of capillary action along the z-direction to the detection zone and/or the control zone, and manifests a signal development in parallel. The dimensions of the assay device along the x and y directions, defining a surface area, on either the first and second faces can vary depending on a desired operational function or an appearance or designed manifestation of the detection zone. The dimensions of assay device along the z-direction can vary depending on the thickness of the substrate or absorbent article into which the device is incorporated. The three-dimensional assay device can be used as a hydration/dehydration sensor that can be easily integrated into an absorbent article. The device allows one to sample from the inner side of the product and reading from the outer side without the need to either open or strip off the product.

In another aspect, the present invention also relates to an absorbent article incorporating a three-dimensional fluidic assay device or sensor. The absorbent article has an inner liner, an absorbent core, an outer cover, and at least one three-dimensional sensor. The device spans the inner liner through the absorbent core to the outer cover. The fluidic assay device further has a first face oriented toward the inner liner, and has a sample deposition zone. The fluidic assay device has a second face oriented away from the absorbent core toward the outer cover. The second face has a detection zone, and may have a control zone, observable from outside of the outer cover. A buffer zone is situated between the surfaces of the first and second faces. A variety of flow-control devices or mechanism may be arranged along the z-direction of the sensor, through the body thickness of the absorbent article, to modulate or regulate the lateral flow of urine or other fluids as the fluid progresses from the deposition zone of the first or inner face to the detection zone of the second or outer face. The mechanisms may take, for example, the form of micro-channels arranged in predetermined patterns and/or one or a number of differentiated substrate densities. These mechanisms may be oriented either parallel or orthogonal to the flow path of fluid along the z-direction.

Alternatively, the device has a porous body with a first planar surface and a second planar surface remote from and oriented either at an angle to or substantially parallel with said first planar surface, as integrated as a part of an absorbent article, such that the device and/or it porous body spans from an inner liner, across an absorbent core, to an outer cover. The second planar surface can be configured to have a variety of designs or patterns to show a visually observable signal. For instance, the signal may manifest in a design as either an appearing or disappearing graphical image, such as a cartoon face (e.g., happy or sad clown) or geometric forms (e.g., circle, square, rectangle, stars, or combinations thereof).

Additional features and advantages of the present three-dimensional sensor or assay device and associated absorbent articles containing such a sensor will be described in the following detailed description. It is understood that the foregoing general description and the following details description and examples are merely representative of the invention, and are intended to provide an overview for understanding the invention as claimed.

### BRIEF DESCRIPTION OF FIGURES

FIG. 1 is a three-quarter schematic representation of a generic absorbent article, such as a diaper 20 or a feminine hygiene pad with a three-dimensional sensor 10, according to an embodiment of the present invention shown in partial cut-away, which traverses the thickness of the article between an inner or top sheet 22 and an outer or back sheet 26.

FIG. 2A is a general schematic illustration of the three-dimensional sensor 10 according to the present invention, as show in x-y-z axes. According to the embodiment, the sensor has a sample deposition zone 12, in a first face 13, which is oriented toward a user, a body 14, and a second face 17, with a detection zone 16, and a control 18. The body 14 may have one or more flow-control mechanisms arranged either parallel with or in layers orthogonal 15 to the direction of sample flow.

FIG. 2B is a cross-sectional schematic illustration of a three-dimensional sensor as depicted in FIG. 2A. The three-dimensional sensor is situated between the inner sheet 22 and outer sheet 26, among the interior absorbent padding 24 of an absorbent article.

FIG. 2C is an alternate partial perspective view of the three-dimensional sensor shown in FIG. 2B.

FIG. 3A is a cross-sectional side view of an absorbent article having a three-dimensional sensor according to an embodiment of the invention, with a sample deposition zone 12 at a first surface 11 towards the upper part and detection zone 16 at a second surface 17 towards the lower part of the article as shown.

FIG. 3B is a cross-sectional side view of an absorbent article, like in FIG. 3A, in which the sensor has a number of different layers of flow control mechanisms 15 arranged in an alternate construction of the body 14 between the first surface and the second surface of the absorbent article.

FIGS. 4A-C are alternate embodiments of the present sensor in the form of triangular, circular, octagonal, or star-shaped configurations of the sensor body 14, and deposition zone 12 or detection zone 16.

FIGS. 5A-C are variations of the internal shape of the sensor along the z-direction spanning the thickness of an absorbent from one surface to an opposing surface.

FIGS. 6A-C are schematic representations of different flow-rate control mechanisms; in particular, the relative density or porosity of the filter medium in the sensor body. Each dot representing either a concave pocket or convex nodule.

FIGS. 7A-D are enlarged schematic representations of flow-rate control mechanisms having a micro-channel pattern to direct the sample through the sensor body.

FIG. 7E is an enlarged schematic representation of a combination of density or porosity gradient and micro-channel pattern.

FIG. 8 is a representation of an alternate micro-channel pattern design.

FIGS. 9A and 9B are representations of another alternate micro-channel pattern design. FIG. 9B shows an amount of fluid beginning to enter and pass through the micro-channel.

### DETAILED DESCRIPTION OF THE INVENTION

Conventional urine testing devices, such as dipsticks or test strips, are oriented horizontally on a largely flat, lateral-flow substrate. Typically the test strip operates by applying a sample at one end of the strip, which wicks to another area of the strip where the sample reacts with chemical agents, and then to a location where a signal can be detected. Alternatively, the test strip is dipped in a sample and taken out quickly, and any color change is then read. Such test device forms can be incorporated only into the inner side of an absorbent product for sampling and signal reading; hence, requiring the user to take-off the product before reading the result signal. This configuration can be an inconvenience, and prevent detecting or monitoring hydration results from outside the absorbent article for monitoring a health condition.

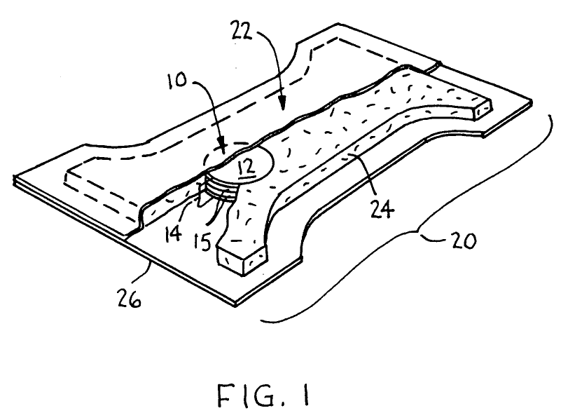

Unlike previously developed lateral flow hydration test where the detection zone and feedback zone are laid in such a way that the sample flows sequentially and laterally to contact the two zones to activate them, the current invention uses a vertical flow scheme to allow the sample contact with either the detection zone or the feedback zone or both simultaneously. This device is a breakthrough\development of a lateral flow dehydration test that, for the first time, has addressed those problems and makes it practical for such a test to be directly incorporated into an absorbent product. FIG. 1 shows an absorbent article 20 with partial cut-away of the absorbent core material 24. According to the invention, the present dehydration/hydration test device or sensor 10 can be placed vertically-oriented to span the thickness of the article between an inner top sheet 22 and an outer back sheet 26. Currently, no such a product is available due to technical difficulty of integrating hydration sensors in absorbent articles where constant monitoring is not feasible.

### I. Three-Dimensional Assay Device

The current invention can address the problem of integrating a test device into an absorbent product, while at the same time retaining certain performance features required for the sensor and absorbent article. Conventional urine test reagent strips do not have a mechanism by which urine volume and electrolyte reactivity can be determined in an effective manner that is not cost prohibitive. The present invention uses technology that encompasses a capillary flow format and has performance features suited for incorporation into absorbent articles. The invention can be configured as a vertical hydration sensor that overcomes the problem and retains the performance features required for integration into the panels of bulky or thick absorbent articles, such as depicted in FIG. 1.

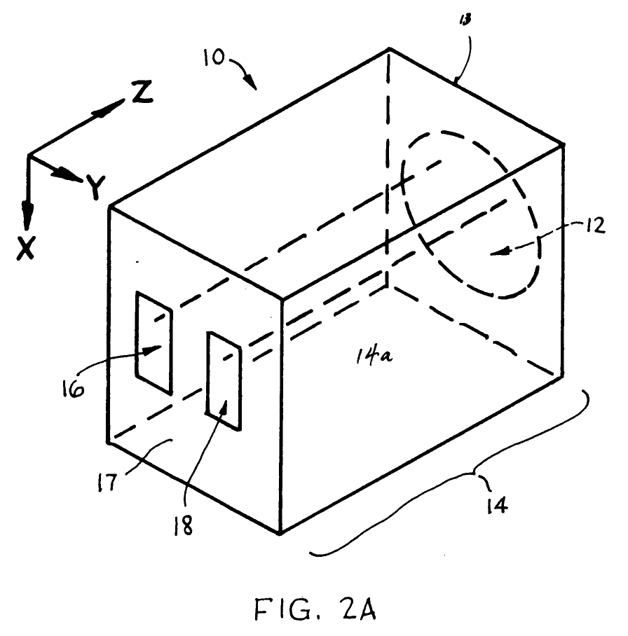

In general, the body of the three-dimensional assay device can be constructed using a chromatographic medium or substrate material that is 1) entirely porous, 2) semi-porous in select areas, 3) porous in some areas and non-porous in other areas, or 4) a combination of materials thereof. The device is integrated as a part of an absorbent article, spanning from an inner liner, across an absorbent core, to an outer cover. According to an embodiment, the substrate can be made of a single medium from the first face to the second face. In other embodiments, the substrate can have more than one layer of different media, each layer adapted to provide a differentiated function or porosity. The sensor body 14 can be unitary such as shown in FIG. 2A, or bifurcated such as in FIGS. 2B or 2C, or otherwise divided into as many zones or display manifestations for results as desire.

FIG. 2A depicts a schematic representation of an example of the present invention that has a sample deposition zone 12 in a first face 13, a buffer zone 14a, and a detection zone 16 in second face 17. As FIGS. 2B and 2C show, from a large single sample deposition zone 12, the body 14 of the assay device or sensor can be bifurcated into two large channels or column-like pathways 19 from the first face to the second face. While the channel itself can be hydrophilic, the area adjacent or surrounding the channel can be made from a material or treated to be hydrophobic. Zones of different hydrophobicity and hydrophilicity can be created through both physical and chemical means. Physical examples may include cutting, etching, laser ablation, etc., and chemical examples may include printing with inks, chemicals, etc.

The fluid can be transported along the z-direction in a column-like pathway that communicates between the first face and second face. The pathway may have channels for the transport of fluids through the substrate from one side to the other. Non-communicative materials that define the boundaries of the channel pathways can be arrange around this fluid transport column to prevent leakage or diffusion of the fluids into adjacent sections of the absorbent article, where the fluid is not desired. A predominate portion of the porous substrate can be fabricated from a variety of cellulose, nylon fibers, or fiber glass. The body of the sensor may be treated or can be innately either hydrophilic or hydrophobic to help conduct a liquid sample and direct transport.

The three-dimensional fluidic assay device can be used to monitor the relative level of hydration in a person. According to an embodiment, the detection zone can be made of a piece of porous membrane that non-diffusively immobilizes a pH indicator. Other reagents may optionally also be included as desired to perform the assay or operational function necessary. The buffer zone includes a porous area, either in the form of a pad or a three-dimensional column or cube, containing buffer components. The buffer agents change in pH as they respond to the different concentration of ions or specific gravity of a urine sample. The sample deposit zone may be configured such that it constitutes either a part of the buffer zone or a separate porous material that can receive a urine sample and subsequently deliver it to the buffer zone. The sample deposit zone is in fluid communication with the buffer zone at one end (e.g., top or upper side) of the buffer zone. The detection zone is in fluid communication with an opposing second end (e.g., bottom or lower side) of the buffer zone to allow the sample to diffuse or flow through to the detection zone by capillary action.

Except for a surface of the sample deposit zone, which is exposed to receive an insult of urine, the other parts of the test device may be enclosed and segregated from its surrounding environment (e.g., absorbent article). This may be accomplished, for instance, by wrapping or sealing the sides of the device with a tape or other impervious films. This sealing is important for the device in a personal care product to prevent sample loss and automatically control the sample volume over time. For the testing, the sample zone receives a urine sample and flows to the buffer zone where the ions in the urine will affect the pH of the buffer in the buffer zone, depending upon the ion concentration. The affected buffer with the sample then flows to the detection zone to trigger a color responding to the final pH of the sample. Another example of the invention is a vertical flow dehydration/hydration test device (see FIG. 2) that consists of a sample zone, a buffer zone, a detection zone and a control zone. The detection zone is made of a piece of porous membrane that non-diffusively immobilizes a pH indicator, optionally other reagents necessary. The immobilized pH indicator in the detection zone will present a different color depending upon the final pH of the samples. The control zone contains a non-diffusively immobilized indicator along with other necessary reagents that changes its color upon contact with a sample no matter what pH the sample has. The buffer zone consists of a porous pad that contains buffer components that change pH responding to the different of ion concentration or specific gravity of urine. The sample zone may be a part of the buffer zone or a separate porous material that can receive a urine sample and subsequently deliver it to the buffer zone. The sample zone is in fluid communication with the buffer zone on the top side of the buffer zone. The detection zone and the control zone are in fluid communication with the bottom side of the buffer zone to allow the sample to flow through capillary action to the detection zone and control zone. The detection zone and the control zone are physically separated. The test device may be wrapped or sealed by a tape or other films except the sample receiving zone. For the testing, the sample zone receives a urine sample and flows to the buffer zone where the ions in the urine will affect the pH of the buffer in the buffer zone, depending upon the ion concentration. The affected buffer with the sample then flows to the detection zone to trigger a color responding to the final pH of the sample. At the same time, the sample also triggers a color for the control zone, indicating the sample has reached the detection zone and the device has received enough sample for the testing. Unlike commercial dipsticks in that all the reagents including buffering reagents and color indicator are diffusively deposited on a porous matrix pad all together, the test devices described here instead have at least two components that are deposited with those reagents and color indicators separately.

Another significant difference is that one of the major reagents, such as color indicator, is non-diffusively immobilized in the detection zone and control zone. The detection zone or control zone may contain both indicator and buffering reagent, where the buffering reagent may be the same or different from the buffering reagent in the buffering component, to adjust the initial color of the zone. The buffering components may or may not be non-diffusively immobilized. The buffering strip is deposited with partially neutralized weak basic or weak acidic polyelectrolytes, either diffusively or non-diffusively. It may be deposited with other buffering systems. Furthermore, a significant portion of the device, including the detection zone and control zone, is sealed in a casing (e.g., using tape) except the sampling zone to prevent or minimize urine loss over time. In addition, a control indicator pad or zone may be created next to the detection zone to confirm that urine sample has been contacted with the detection zone and the testing has been done properly. Examples of pH indicators for the detection zone include any dyes that change color responding to pH change or ion concentration changes or ion strength change, such as bromothymol blue or thymol blue. Examples of porous membranes to immobilize the pH indicator include any non-charged and charged porous materials. Examples of indicators for the control zone include any dyes that change color upon contact with urine no matter what pH it has. Examples of the indicators include bromophenol blue, bromcresol green and Congo red. The charged porous materials used to immobilized indicators in the detection zone and control zone include either positively charged, negatively charged or zwitterionic nature, such as Biodyne A, B, C or plus membranes of Pall Co. Examples of the buffering materials include weak acidic polyelectrolytes and weak basic polyelectrolytes, such as poly(acrylic acid), poly(maleic acid), poly(vinylamine) and poly(4-vinylpyridine). The buffering materials are at least partially neutralized, e.g., 50% neutralized. Examples of porous matrices for the buffering components include cellulose materials, glass fiber materials and nonwoven materials. The shapes and the dimensions of the various zones can be varied. However, the detection zone and control zone are preferred to be thin. The devices have many advantages over commercial tests for specific gravity. First, the devices provide stable color signals over a long period of time (6 hours) while commercial devices require immediate readings (within 2 minutes) because the color signals are not stable. The stable color signals are achieved through non-diffusive immobilization of the color indicator (no leaching) and minimization of sample evaporation (sealing). Secondly, the devices are cased to minimize reagent exposures to users. Thirdly, a separate indicator signal can be integrated into the devices to provide feedbacks to users for assurance. Furthermore, the physical separation of color indicator and buffering components allow easy tailoring to reduce impact of sample loss and diffusion over time on pH fluctuation around the immobilized indicator. Those features are critical for the devices to be integrated in an absorbent article. Currently available commercial devices lack those features.

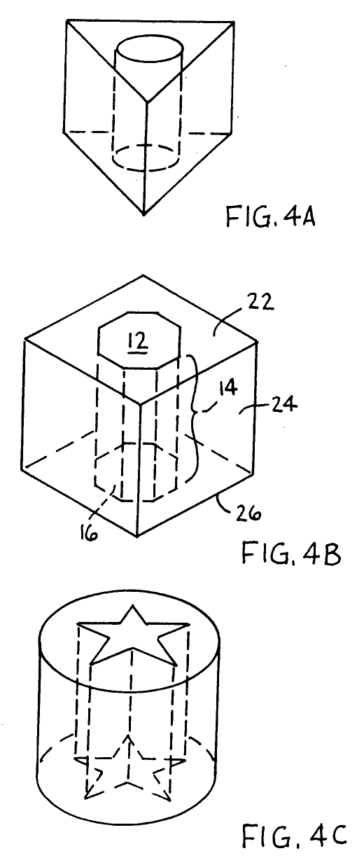

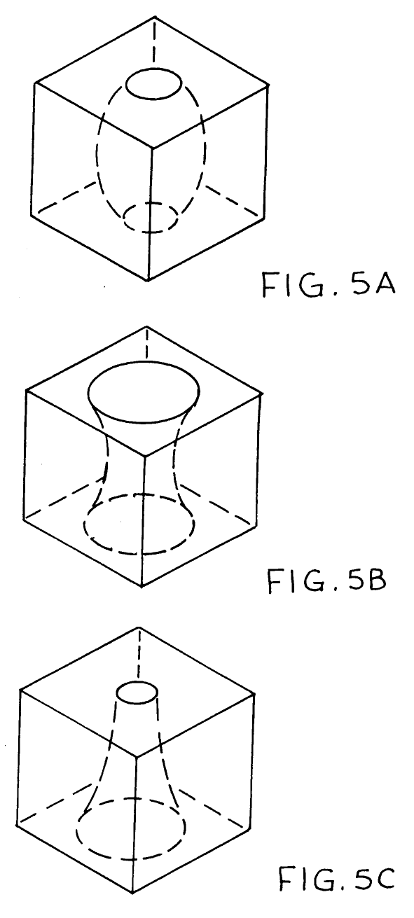

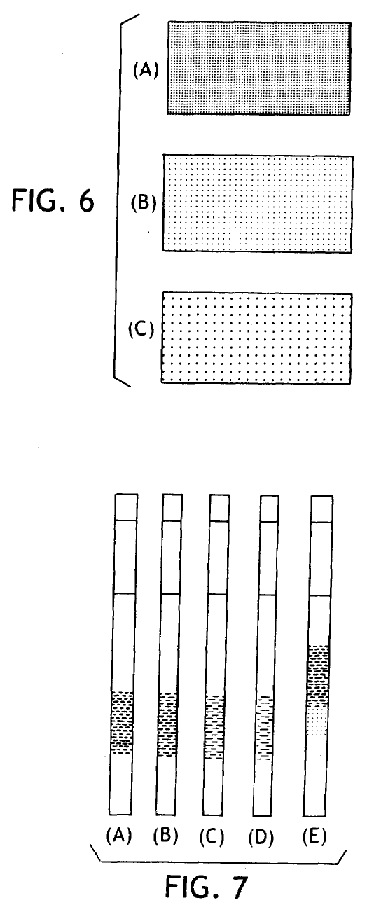

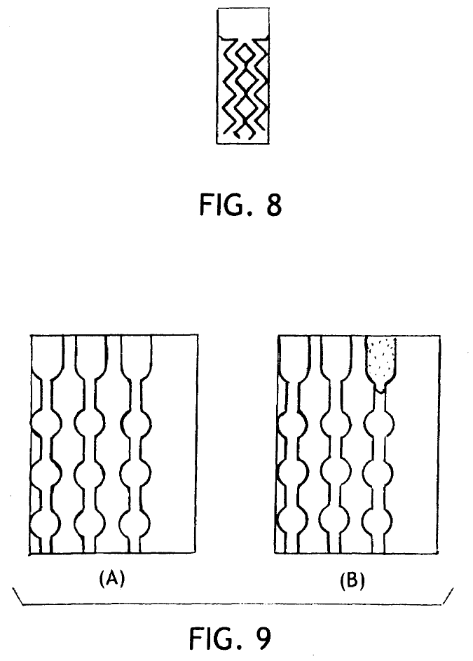

For modifying or controlling the rate of slow of a urine sample through the sensor, a number of different mechanisms 15 may be employed. For example, a number of vertically or horizontally oriented flow-control devices, such as designs of micro-channel patterns, density or gradient scales, or filters, such as represented in FIGS. 6-8, can be incorporated in the body of each sensor unit to help regulate the flow rate of the liquid from one side to the other along the z-direction of the sensor unit. Vertically oriented flow rate control devices can be placed to run parallel with the z-direction of the liquid flow, such that the liquid can travel largely along one plane from the deposition zone to detection zone, such as FIGS. 7-9. In contrast, horizontally oriented flow rate control devices can be situated largely orthogonal to the liquid flow direction such that the liquid passes through the plane of each horizontal layer, such as depicted in FIGS. 1, 2B, 2C, or 3B. Laminations can include various types of tapes and polymer films. The present sensor device can include a large number or combination of layers, each with a particular physical or chemical property, as long as the layers are in fluid contact with each other.

The physical dimensions of the sensor can vary depending on the desired application and absorbent needs. Physical dimensions along x-y directions, defining a plane, can be any size; a practical size for this specific application would be limited to the desired inner wetting area or outer displayable surface area of the absorbent article. Physical dimensions along the z-direction also can be any size, but will be limited typically to the thickness of the absorbent article. For instance, possible dimensions along the x-y direction can range from as short as about 1 mm to as long as about 45 cm. Typically, the length dimensions are between about 2 or 3 cm up to about 20 or 25 cm (e.g., about 5, 8, 10, 12, 15, or 17 cm) for each side as desired. The first and/or second faces each can have overall dimensions of about 2-8 cm², about 10-25 cm², or about 16-64 cm², or 91-400 c m², as desired. The dimension along the z-direction may range from about 0.10 or 0.20 cm to about 10 or 15 cm (e.g., 1-3 cm, 2-5 cm, 3-8 cm, or 4-7 cm).

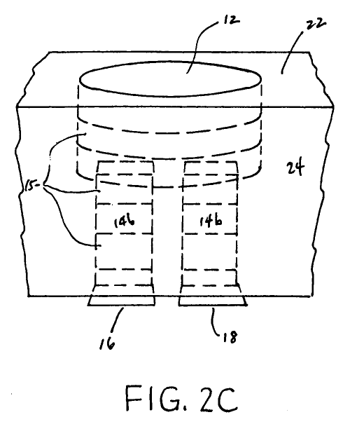

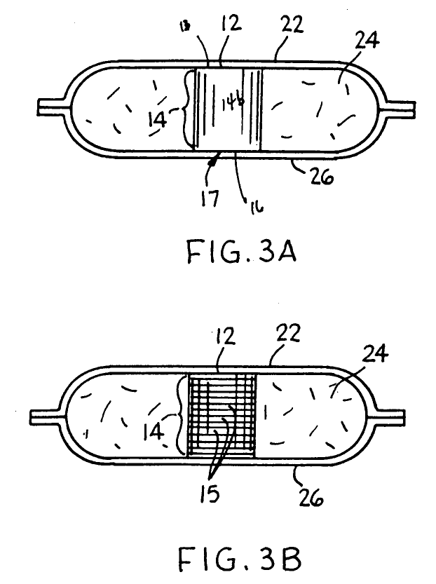

A sensor according to the present invention can take the form of a variety of shapes or designs. The face of the sensor or the deposition zone that is nearer to the user can have either an octagonal, rectangular, square, round, oval, hour-glass-like, saddle-like, star-like, or triangular shape, such as illustrated in FIGS. 4A-C. The first surface desirably should be sufficiently large to enable a liquid sample to contact in sufficient amounts to wet the deposition zone thoroughly and permit the liquid to wick into the interior of the sensor. The second face may also include at least one control zone. The porous substrate has a three-dimensional geometric form with at least two surfaces. The signal development in said detection zone and said control zone are parallel, and in some cases simultaneous. Alternatively, three-dimensional geometric form has at least three surfaces, with at least two of the surfaces being either adjacent to or opposing another surface. In other examples, the three-dimensional geometric form may be selected from a rectilinear polygon or a curvilinear polygon. For instance, the geometric form can be a cube-like or other rectilinear shape, a curved or flat-sided cylinder, a barrel-like shape (i.e., truncated ellipsoid), a sphere, a cone, or frustum of any one of the preceding forms (e.g., frusto-conical or frusto-pyramidal shape), such as illustrated in FIGS. 5A-C, or can be any shape.

In certain embodiments, the three-dimensional fluidic assay device can have a unitary or monolithic consistency. That is it is composed of one material or has a uniform porosity throughout. In other embodiments, the device can have different regions or sections, each with a different gradient or degree of porosity, or hydrophilic or hydrophobic characteristic. For instance, a central portion of the device can function like a chromatographic column, which is more porous and/or hydrophilic than the surrounding section. This column constitutes one or more pathways along the z-direction of the device through which a liquid sample is transported from the deposition zone to the detection zone. An area, either adjacent or surrounding each of the columnar pathways, can be fabricated with a material that is hydrophobic or modified to be hydrophobic. This feature can help channel and direct the flow of sample as it migrates through the three-dimensional sensor device. Along the z-direction a buffer zone can be situated between the first and second faces. Moreover in some embodiments, a flow rate control zone may be constructed between the first and second faces along the z-direction. The flow rate control zone may have a gradient of porous media. In some examples, the porous substrate body of the device comprises a laminate structure of at least two layers orthogonally oriented to said z-direction. In other examples, more than two layers may be included.

It is envisioned that the sensor device can operate similar to a chromatographic column. In general, the chromatographic medium 14b can be made from any of a variety of materials through which the urine is capable of passing. For example, the chromatographic medium 14b can be a porous membrane formed from synthetic or naturally occurring materials, such as polysaccharides (e.g., cellulose materials such as paper and cellulose derivatives, such as cellulose acetate and nitrocellulose); polyether sulfone; polyethylene; nylon; polyvinylidene fluoride (PVDF); polyester; polypropylene; silica; inorganic materials, such as deactivated alumina, diatomaceous earth, MgSO₄, or other inorganic finely divided material uniformly dispersed in a porous polymer matrix, with polymers such as vinyl chloride, vinyl chloride-propylene copolymer, and vinyl chloride-vinyl acetate copolymer; cloth, both naturally occurring (e.g., cotton) and synthetic (e.g., nylon or rayon); porous gels, such as silica gel, agarose, dextran, and gelatin; polymeric films, such as polyacrylamide; and so forth.

The assay device may include (but not necessary) a semi-rigid support materials. Examples of suitable materials for the support include, but are not limited to, glass, polymeric materials, such as polystyrene, polypropylene, polyester (e.g., Mylar® film), polybutadiene, polyvinylchloride, polyamide, polycarbonate, epoxides, methacrylates, and polymelamine; and so forth. To provide a sufficient structural backing for the chromatographic medium, the support is generally selected to have a certain minimum thickness. Thus, for example, the support can have a thickness that ranges from about 100 to about 5,000 micrometers, in some embodiments from about 150 to about 2,000 micrometers, and in some embodiments, from about 250 to about 1,000 micrometers.

### II. Absorbent Article

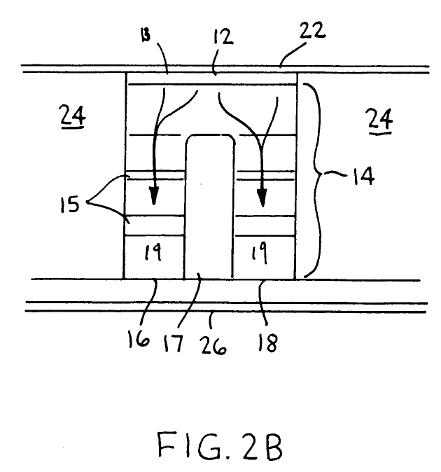

The invention also discloses an absorbent article that contains the three-dimensional assay devices. FIG. 1 illustrates a layout of the device in an absorbent article according to an embodiment. The device is inserted through the absorbent core so that the sample receiving zone is exposed to the inner surface of the absorbent article and the detection zone and/or the control zone are exposed to the outer cover to permit possible direct reading of a signal from the outer cover. The absorbent article can be an insert that can be attached to an absorbent article. FIGS. 3A and 3B show cross-sectional views of the absorbent article with the sensor device spanning the absorbent core from an inner top sheet to an outer film layer.

According to the present invention, an “absorbent article” generally refers to any article capable of absorbing water or other fluids. Examples of some absorbent articles include, but are not limited to, personal care absorbent articles, such as diapers, training pants, absorbent underpants, adult incontinence articles, feminine hygiene products (e.g., sanitary napkins), swim wear, baby wipes, etc.; medical absorbent articles, such as garments, fenestration materials, underzones, bedzones, bandages, absorbent drapes, and medical wipes; food service wipers; clothing articles, etc. Materials and processes suitable for forming such absorbent articles are familiar to those skilled in the art. Typically, absorbent articles include a substantially liquid-impermeable layer (e.g., outer cover), a liquid-permeable layer (e.g., inner or body-side liner, surge layer, etc.), and an absorbent core disposed between the two.

Various embodiments of an absorbent article that may incorporate the three-dimensional assay device according to the present invention will now be described in more detail. For purposes of illustration and discussion only, the absorbent article shown in FIG. 1, can be a diaper 20. In the illustrated embodiment, the diaper 20 is shown as having an hourglass-like shape in an unfastened configuration. However, other shapes can of course be utilized, such as a generally rectangular shape, T-shape, or I-shape. Although not shown in detail, the diaper 20 may include a chassis formed by various components, including an inner body-side liner 22, absorbent core 24, outer cover or film layer 26, and surge layer. (It should be understood, however, that other layers can also be used in exemplary embodiments of the present invention.) Likewise, one or more of the layers referred to in FIG. 1 can also be eliminated in certain exemplary embodiments of the present invention.

The body-side liner 22 is generally employed to help isolate the wearer's skin from liquids held in the absorbent core. For example, the liner presents a body-facing surface that is typically compliant, soft feeling, and non-irritating to the wearer's skin. Typically, the liner is also less hydrophilic than the absorbent core 24 so that its surface remains relatively dry to the wearer. As indicated above, the liner can be liquid-permeable to permit liquid to readily penetrate through its thickness. Exemplary liner constructions that contain a nonwoven web are described in U.S. Patent No. 5,192,606 to Proxmire, et al.; U.S. Pat. No. 5,702,377 to Collier, IV, et al.; U.S. Pat. No. 5,931,823 to Stokes, et al.; U.S. Pat. No. 6,060,638 to Paul, et al.; and U.S. Pat. No. 6,150,002 to Varona, as well as U.S. Patent Application Publication Nos. 2004/0102750 to Jameson; 2005/0054255 to Morman, et al.; and 2005/0059941 to Baldwin, et al., all of which are incorporated herein in their entirety by reference thereto for all purposes.

The diaper can also include a surge layer that helps to decelerate and diffuse surges or gushes of liquid that can be rapidly introduced into the absorbent core. Desirably, the surge layer rapidly accepts and temporarily holds the liquid prior to releasing it into the storage or retention portions of the absorbent core. In the illustrated embodiment, for example, the surge layer is interposed between an inwardly facing surface of the body-side liner and the absorbent core. Alternatively, the surge layer can be located on an outwardly facing surface of the body-side liner. The surge layer is typically constructed from highly liquid-permeable materials. Examples of suitable surge layers are described in U.S. Pat. No. 5,486,166 to Ellis, et al. and U.S. Pat. No. 5,490,846 to Ellis, et al., which are incorporated herein in their entirety by reference thereto for all purposes.

The outer cover sheet 26 is typically formed from a material that is substantially impermeable to liquids. For example, the outer cover can be formed from a thin plastic film or other flexible liquid-impermeable material. In one embodiment, the outer cover 26 is formed from a polyethylene film having a thickness of from about 0.01 millimeter to about 0.05 millimeter. The film can be impermeable to liquids, but permeable to gases and water vapor (i.e., “breathable”). This permits vapors to escape from the absorbent core 24, but still prevents liquid exudates from passing through the outer cover 26. If a more cloth-like feeling is desired, the outer cover 26 can be formed from a polyolefin film laminated to a nonwoven web. For example, a stretch-thinned polypropylene film can be thermally laminated to a spunbond web of polypropylene fibers.

Besides the above-mentioned components, the diaper can also contain various other components. For example, the diaper can also contain a substantially hydrophilic tissue wrapsheet (not illustrated) that helps maintain the integrity of the fibrous structure of the absorbent core. The tissue wrapsheet is typically placed about the absorbent core over at least the two major facing surfaces thereof, and composed of an absorbent cellulosic material, such as creped wadding or a high wet-strength tissue. The tissue wrapsheet can be configured to provide a wicking layer that helps to rapidly distribute liquid over the mass of absorbent fibers of the absorbent core. The wrapsheet material on one side of the absorbent fibrous mass can be bonded to the wrapsheet located on the opposite side of the fibrous mass to effectively entrap the absorbent core. Furthermore, the diaper can also include a ventilation layer (not shown) that is positioned between the absorbent core and the outer cover. When utilized, the ventilation layer can help insulate the outer cover from the absorbent core, thereby reducing dampness in the outer cover. Examples of such ventilation layers can include a nonwoven web laminated to a breathable film, such as described in U.S. Pat. No. 6,663,611 to Blaney, et al., which is incorporated herein in its entirety by reference thereto for all purposes.

In some embodiments, the diaper can also include a pair of side panels (or ears) (not shown) that extend from the side edges of the diaper into one of the waist regions. The side panels can be integrally formed with a selected diaper component. For example, the side panels can be integrally formed with the outer cover or from the material employed to provide the top surface. In alternative configurations, the side panels can be provided by members connected and assembled to the outer cover, the top surface, between the outer cover and top surface, or in various other configurations. If desired, the side panels can be elasticized or otherwise rendered elastomeric by use of the elastic nonwoven composite of the present invention. Examples of absorbent articles that include elasticized side panels and selectively configured fastener tabs are described in PCT Patent Application WO 95/16425 to Roessler; U.S. Pat. No. 5,399,219 to Roessler et al.; U.S. Pat. No. 5,540,796 to Fries; and U.S. Pat. No. 5,595,618 to Fries, each of which is incorporated herein in its entirety by reference thereto for all purposes.

The diaper can also include a pair of containment flaps that are configured to provide a barrier and to contain the lateral flow of body exudates. The containment flaps 112 can be located along the laterally opposed side edges of the bodyside liner 105 adjacent the side edges of the absorbent core. The containment flaps can extend longitudinally along the entire length of the absorbent core, or can only extend partially along the length of the absorbent core. When the containment flaps are shorter in length than the absorbent core 103, they can be selectively positioned anywhere along the side edges of diaper in a crotch region. In one embodiment, the containment flaps extend along the entire length of the absorbent core to better contain the body exudates. Such containment flaps are generally well known to those skilled in the art. For example, suitable constructions and arrangements for the containment flaps are described in U.S. Pat. No. 4,704,116 to Enloe, which is incorporated herein in its entirety by reference thereto for all purposes.

To provide improved fit and to help reduce leakage of body exudates, the diaper can be elasticized with suitable elastic members, as further explained below. For example, the diaper can include leg elastics constructed to tension operably the side margins of the diaper to provide elasticized leg bands which can closely fit around the legs of the wearer to reduce leakage and provide improved comfort and appearance. Waist elastics can also be employed to elasticize the end margins of the diaper to provide elasticized waistbands. The waist elastics are configured to provide a resilient, comfortably close fit around the waist of the wearer.

The diaper can also include one or more fasteners. For example, two flexible fasteners on opposite side edges of waist regions create a waist opening and a pair of leg openings about the wearer. The shape of the fasteners can generally vary, but can include, for instance, generally rectangular shapes, square shapes, circular shapes, triangular shapes, oval shapes, linear shapes, and so forth. The fasteners can include, for instance, a hook-and-loop material, buttons, pins, snaps, adhesive tape fasteners, cohesives, fabric-and-loop fasteners, etc. In one particular embodiment, each fastener includes a separate piece of hook material affixed to the inside surface of a flexible backing.

The various regions and/or components of the diaper can be assembled together using any known attachment mechanism, such as adhesive, ultrasonic, thermal bonds, etc. Suitable adhesives can include, for instance, hot melt adhesives, pressure-sensitive adhesives, and so forth. When utilized, the adhesive can be applied as a uniform layer, a patterned layer, a sprayed pattern, or any of separate lines, swirls or dots. In the illustrated embodiment, for example, the outer cover and body-side liner are assembled to each other and to the absorbent core using an adhesive. Alternatively, the absorbent core can be connected to the outer cover using conventional fasteners, such as buttons, hook and loop-type fasteners, adhesive tape fasteners, and so forth. Similarly, other diaper components, such as the leg elastic members, waist elastic members and fasteners, can also be assembled into the diaper using any attachment mechanism.

Generally speaking, the devices of the present disclosure can be incorporated into the absorbent article in a variety of different orientations and configurations, so long as the device is capable of receiving urine and providing a signal to a user or caregiver to convey the urine specific gravity. For example, the sampling zone and control zone can be visible to the user or caregiver so that a simple, accurate, and rapid indication can be provided. The visibility of such layer(s) can be accomplished in a variety of ways. For example, in some embodiments, the absorbent article can include a transparent or translucent portion (e.g., window, film, etc.) that allows the sample detection zone and/or control zone to be readily viewed without removal of the absorbent article from the wearer and/or without disassembly of the absorbent article. In other embodiments, the detection zone and/or control zone can extend through a hole or aperture in the absorbent article for observation. In still other embodiments, the detection zone and/or control zone can simply be positioned on a surface of the absorbent article for observation.

Regardless of the particular manner in which it is integrated, urine can be directly discharged to a portion of the sampling zone, a liquid permeable cover or other material surrounding assay device, or can be discharged onto a component of the absorbent article into which the assay device has been integrated.

After a sufficient reaction time, the intensity of the color can be measured to quantitatively or semi-quantitatively determine the urine volume and/or the urine specific gravity results. Nevertheless, while quantitative testing can be performed, qualitative testing is typically employed to provide early testing and monitoring of a health condition. Thus, when a certain urine volume and/or urine specific gravity is detected, the user or caregiver is given an indication that further quantitative testing can be undertaken. For example, a diaper having an integrated assay device can be periodically used with infants or non-ambulatory patients as part of a monitoring program that tests for urine volume and/or urine specific gravity. Upon indication of a sufficiently low urine volume and/or high urine specific gravity, further quantitative testing can then be undertaken to determine the scope and stage of the problem detected so as to provide additional treatment information.

### III. Examples

The present invention can be better understood with reference to the following example.
1. Preparation of detection zone (pad): Biodyne plus membrane from Pall Co. was soaked with bromothymol blue in water and dried at room temperature.
2. Preparation of control zone (pad): Biodyne plus membrane from Pall Co. was soaked with bromocresol green and citric acid in water and dried at room temperature.
3. Preparation of buffering zone (pad): Pall's cellulose pad (5 mm thick) was soaked with polyacrylic acid buffer (pH=8.1) and dried at room temperature.
4. Assembling of devices with detection zone: Two pieces of buffer pads were stacked together by a tape. A piece of a detection pad was laminated at the bottom of the stacked buffer pad. The whole device was sealed by a tape except the top part of the device to receive a sample.
5. Assembling of devices with detection zone and control zone: Two pieces of buffer pads were stacked together by a tape. A piece of a detection pad was laminated on to a portion of the bottom of the stacked buffer pad. A piece of a control pad was laminated on to another portion of the bottom of the stacked buffer pad. The whole device was sealed by a tape except the top part of the device.
6. Testing the devices made in step 5, above: 1 ml synthetic urine sample with a different specific gravity, ranging from 1.002, 1.008, 1.014, 1.020, 1.025 and 1.030, was applied to each device. Two minutes later, the color of the detection zone shows green, green, green, yellow/green, yellow and yellow, respectively. The signals are stable for more than 6 hours.

The present invention has been described both generally and in detail by way of examples and the figures. Persons skilled in the art, however, can appreciate that the invention is not limited necessarily to the embodiments specifically disclosed, but that substitutions, modifications, and variations may be made to the present invention and its uses without departing from the spirit and scope of the invention. Therefore, changes should be construed as included herein unless the modifications otherwise depart from the scope of the present invention as defined in the following claims.

We claim:

1. A three-dimensional fluidic assay device comprising a porous substrate with a first face or plane defined along x-y coordinate axes, and a second face remote in a z-direction from said first face; said first face having a sample deposition zone, said second face having at least one detection zone, such that when a sample is deposited in said sample deposition zone, fluid is transported by means of capillary action along said z-direction to said detection zone and manifests a signal development.

2. The three-dimensional fluidic assay device according to claim 1, wherein said second face also includes at least one control zone

3. The three-dimensional fluidic assay device according to claim 1, wherein said signal development in said detection zone and said control zone are parallel

4. The three-dimensional fluidic assay device according to claim 3, wherein said signal development is simultaneously.

5. The three-dimensional fluidic assay device according to claim 1, wherein said fluid is transported along said z-direction in column-like pathways that communicate between said first face and said second face.

6. The three-dimensional fluidic assay device according to claim 1, wherein said column-like pathways are hydrophilic.

7. The three-dimensional fluidic assay device according to claim 1, wherein an area either adjacent or surrounding each of said column-like pathways is hydrophobic.

8. The three-dimensional fluidic assay device according to claim 1, wherein along said z-direction a buffer zone is situated between said first face and second face.

9. The three-dimensional fluidic assay device according to claim 1, wherein along said z-direction is a flow rate control zone, between said first face and second face.

10. The three-dimensional fluidic assay device according to claim 1, wherein said flow rate control zone encompasses a gradient of porous media from said second surface to said first surface.

11. The three-dimensional fluidic assay device according to claim 1, wherein said porous substrate comprises a laminate structure of at least two layers orthogonally oriented to said z-direction.

12. The three-dimensional fluidic assay device according to claim 1, wherein said porous substrate has a three-dimensional geometric form with at least two surfaces.

13. The three-dimensional fluidic assay devices according to claim 12, wherein said three-dimensional geometric form has at least three surfaces, with at least two of said surfaces being either adjacent to or opposing another surface.

14. The three-dimensional fluidic assay device according to claim 12, wherein said three-dimensional geometric form is selected from a rectilinear polygon or a curvilinear polygon.

15. The three-dimensional fluidic assay device according to claim 12, wherein said three-dimensional geometric form is selected from a cube-like shape, a cylinder, a truncated ellipsoid, a cone, or a frusto-conical, or frusto-pyramidal shape.

16. The three-dimensional fluidic assay device according to claim 1, wherein said device is a monitor for relative level of hydration in a person.

17. The three-dimensional fluid assay device according to claim 1, wherein said device is adapted to employ at least one type of the following detection modes: immunoassay, enzymatic assay, or small molecule assay.

18. The three-dimensional fluidic assay device according to claim 1, wherein said device is integrated as a part of an absorbent article, spanning from an inner liner, across an absorbent core, to an outer cover.

19. An absorbent article comprising: an inner liner, an absorbent core, an outer cover, and at least one three-dimensional fluidic assay device that spans said inner liner through said absorbent core to said outer cover; said fluidic assay device having a first face oriented toward said inner liner, said first face having a sample deposition zone, said fluidic assay device further having a second face oriented away from said absorbent core toward said outer cover, said second face having a detection zone and a control zone observable from outside of said outer cover; and a buffer zone is situated between said first and second faces.

20. The absorbent article according to claim 17, wherein said article is a diaper, training pants, absorbent underpants, a feminine hygiene product, adult incontinence articles, medical fenestration materials, bandages, absorbent drapes, and medical wipes, food service wipers, or clothing articles.

21. The absorbent article according to claim 17, wherein said first face is defined along x-y coordinate axes, and said second face is remote in a z-direction from said first face.

22. The absorbent article according to claim 17, wherein a fluid is transported by means of capillary action along said z-direction to said detection zone and manifests a signal development.

23. The absorbent article according to claim 17, wherein a signal development in said detection zone and said control zone are parallel.

24. The absorbent article according to claim 20, wherein said fluid is transported along said z-direction in column-like pathways that communicate between said first face and said second face.

25. The absorbent article according to claim 17, wherein said column-like pathways are hydrophilic.

26. The absorbent article according to claim 17, wherein an area either adjacent or surrounding each of said column-like pathways is hydrophobic.

27. The absorbent article according to claim 17, wherein along said z-direction a buffer zone is situated between said first face and second face.

28. The absorbent article according to claim 17, wherein along said z-direction is a flow rate control zone, between said first face and second face.

29. The absorbent article according to claim 26, wherein said flow rate control zone encompasses a device or gradient of porous media from said second surface to said first surface.

30. The absorbent article according to claim 17, wherein said flow rate control zone device is oriented vertically to run parallel with the z-direction.

31. The absorbent article according to claim 17, wherein said flow rate control zone device is oriented horizontally and orthogonal to the z-direction.

32. The absorbent article according to claim 17, wherein said flow rate control zone device functions as a chromatographic column.

33. A fluidic assay device for monitoring relative hydration levels in a person, the device comprising: a porous body with a first planar surface and a second planar surface remote from and oriented either at an angle to or substantially parallel with said first planar surface; said first face having a sample deposition zone, said second planar surface having at least one detection zone and optionally a control zone, such that when a sample is deposited in said sample deposition zone, fluid is transported by means of capillary action from said first planar surface to said second planar surface through said porous body to said detection zone and manifests a signal development, and said device is integrated as a part of an absorbent article, spanning from an inner liner, across an absorbent core, to an outer cover

34. The fluidic assay device according to claim 33, wherein said device is adapted to employ at least one type of the following detection modes: immunoassay, enzymatic assay, or small molecule assay.
